(19) United States
(12) Patent Application Publication
(10) Pub. No.: US 2019/0011421 A1
(43) Pub. Date: Jan. 10, 2019

(54) SYSTEMS AND METHODS FOR SUBSTANCE DETECTION USING DOPED MEMBRANES

(71) Applicant: Morpho Detection, LLC, Newark, CA (US)

(72) Inventors: William Rogers, Somerville, MA (US); Vladimir Romanov, Pelham, NH (US); Stefan Lukow, Hampstead, NH (US); Hartwig Schmidt, Tewksbury, MA (US)

(21) Appl. No.: 15/643,169

(22) Filed: Jul. 6, 2017

(51) Int. Cl. G01N 33/22; G01N 27/62

(52) U.S. Cl. CPC G01N 33/227; G01N 27/622

(57) ABSTRACT

The present disclosure is directed to methods and systems for detecting a substance of interest. The methods and systems include contacting the substance of interest with a doped membrane, the doped membrane comprising at least one semi-permeable medium doped with at least one acid. The systems and methods further include desorbing the doped membrane to release the substance of interest, performing an analysis of the substance of interest, and detecting the substance of interest.

### BACKGROUND OF THE DISCLOSURE

The embodiments described herein relate generally to detection techniques for chemical substances, and, more particularly, to contacting a doped membrane with a substance of interest, thereby increasing the detection sensitivity and/or selectivity for the substance of interest. More specifically, the methods and systems include contacting a substance of interest with a doped membrane comprising at least one semi-permeable medium doped with an acid. The systems and methods further include desorbing the doped membrane to release the substance of interest and performing an analysis on the substance of interest to detect the substance of interest.

Certain substances of interest (e.g., narcotics, energetic materials, explosives such as home-made explosive (HMEs)) have low vapor pressures and high melting points, making their detection a challenge using conventional trace detection systems and methods. In some instances, it is desirable to increase the volatility of the substance of interest (such as through chemical modification) for improved detection. For safety reasons, it is desirable to provide methods and systems for increased substance of interest volatility that presents minimal chemical exposure risk to users/operators and other contacted items. In some instances, it is additionally advantageous to increase substance of interest volatility without requiring significant alteration or adaptation of conventional trace detection system hardware or sample throughput.

There is a need, therefore, for trace detection systems and methods that utilize a sensitive, low-cost approach for detecting substances of interest having low volatilities with little to no modification of instrument hardware or throughput, while minimizing chemical exposure. The present disclosure achieves these benefits by utilizing doped membranes to contact and modify a substance of interest, effectively increasing its volatility and improving detection thereof. In particular, chemical modification for increased volatility of a substance of interest may be achieved through contact with a doped membrane, such as a semi-permeable medium doped with an acid.

### BRIEF DESCRIPTION OF THE DISCLOSURE

In one embodiment of the present disclosure, a device for detecting a substance of interest is disclosed. The device includes a doped membrane, wherein the membrane comprises at least one semi-permeable medium and is doped with at least one acid.

In another embodiment of the present disclosure, a method for detecting a substance of interest is disclosed. The method includes contacting a substance of interest with a doped membrane, wherein the membrane comprises at least one semi-permeable medium and is doped with at least one acid. The method also includes heating the substance of interest and the doped membrane in a desorber. The method further includes performing an analysis of the substance of interest and detecting the substance of interest.

In yet another embodiment of the present disclosure, a system for detecting a substance of interest is disclosed. The system includes an inlet configured to receive a substance of interest. The system also includes a doped membrane, wherein the membrane comprises at least one semi-permeable medium and is doped with at least one acid. The system further includes a desorber and an analysis device coupled in flow communication with the inlet and the desorber, wherein the analysis device is configured to perform an analysis on the substance of interest.

### BRIEF DESCRIPTION OF THE DRAWINGS

FIG. 1 is an exemplary embodiment of reactant ion peak intensities associated with KClO₃ detection in accordance with the present disclosure.

FIG. 2 is an exemplary embodiment of a graphical depiction of the KClO₃ detection response shown in FIG. 1, in accordance with the present disclosure.

FIG. 3A is an exemplary embodiment of a KClO₃ detection response curve associated with an exemplary doped membrane in accordance with the present disclosure. FIG. 3B is an exemplary embodiment of a KClO₃ detection response curve associated with another exemplary doped membrane in accordance with the present disclosure.

FIG. 4 is an exemplary embodiment of reactant ion peak intensities associated with KClO₄ detection in accordance with the present disclosure.

FIG. 5 is an exemplary embodiment of a graphical depiction of KClO₄ detection response shown in FIG. 4, in accordance with the present disclosure.

FIG. 6A is an exemplary embodiment of a KClO₄ detection response curve associated with an exemplary doped membrane in accordance with the present disclosure. FIG. 6B is an exemplary embodiment of a KClO₄ detection response curve associated with another exemplary doped membrane in accordance with the present disclosure.

FIG. 7 is an exemplary embodiment of a block diagram of a trace detection system in accordance with the present disclosure.

FIG. 8 is another exemplary embodiment of a block diagram of a trace detection system in accordance with the present disclosure.

### DETAILED DESCRIPTION OF THE DISCLOSURE

Trace detection systems are utilized for analyzing, detecting, and identifying various substances of interest, such as explosives and narcotics. In some embodiments of the present disclosure, a doped membrane is contacted with the substance of interest to increase detection sensitivity and/or selectivity of the substance of interest by increasing its volatility. In some embodiments, the substance of interest includes at least one of an explosive, an energetic material, a taggant, a narcotic, a toxin, a chemical warfare agent, a biological warfare agent, a pollutant, a pesticide, a toxic industrial chemical, a toxic industrial material, a homemade explosive, a pharmaceutical trace contaminant and combinations thereof. In some embodiments, a substance of interest comprises an inorganic salt such as an inorganic oxidizer salt, which is becoming increasingly prevalent in various home-made explosives (HMEs) and poses a significant detection challenge in trace detection systems that rely on vaporization of the sample or substance of interests for detection, due to their low volatilities and high melting points.

The doped membrane is comprised of at least one semi-permeable medium that is doped with at least one acid. In some embodiments, the membrane is an ion exchange membrane. In some embodiments, the semi-permeable medium is a polymer or copolymer. For example, in some embodiments, the semi-permeable medium is a polybenzimidazole (PBI) copolymer. The at least one acid is an organic acid or an inorganic acid. In some embodiments, the organic acid is at least one of trifluoroacetic acid and formic acid. In some embodiments, the organic acid is a strong organic acid with a low molecular weight. In some embodiments, the inorganic acid is at least one of phosphoric acid and polyphosphoric acid. In some embodiments, the inorganic acid is an inorganic acid having an acidity lower than that of phosphoric acid (i.e., pKa₁ of from about 2.10-2.20, or about 2.14). The semi-permeable medium is doped with the acid at a concentration of from about 0.50 weight percent to about 20 weight percent of the acid. In some embodiments, the semi-permeable medium is doped with the acid at a concentration of from about 1 weight percent to about 5 weight percent of the acid.

Once doped and thermally treated, the acid has a slow diffusion rate at ambient/room temperatures (see Tables 1a and 1b) and is not released from the semi-permeable medium. Consequently, the doped membrane has generally neutral surface pH. For example, the diffusion rates of phosphoric acid in PBI membranes are a function of the water content, temperature and phosphoric acid content of the PBI membrane. The molecular/ionic species that are diffusing in the membrane change with temperature, water content and molar ratio of the phosphoric acid versus the repeated subunit of the polymer, such as benzimidazole units in the PBI membrane. As the water content decreases, phosphoric acid condenses to larger polyphosphoric acids. These are primarily dimers and trimers having higher acidity, higher viscosity, and lower hygroscopicity than that of phosphoric acid.

Because phosphoric acid is a weak acid (pKa of 2.1), a mixture consisting of free acid, polyphosphoric acids and the corresponding ions is present in aqueous solutions. Perchloric acid (pKa−10) and chloric acid (pKa−1) are strong acids with a Ka>1, and completely dissociate in aqueous solution. Generally, protons move to the stronger conjugate base, the phosphate anion, yielding phosphoric acid instead of forming the inorganic oxidant acids. However, if phosphoric acid condensation takes place on the surface during drying of the membrane after doping or during the desorption process, dimers (pyro-phosphoric acid, pKa 0.8 to −0.5) and trimers (tri-phosphoric acid, pKa 0.5 to −0.5) are formed with a much higher acidity, thus pushing the protonation in the direction of inorganic oxidant acid formation.

Removal of hydration water inside the membrane together with a polycondensation step makes water available to solvate or dissolve any inorganic oxidants on the surface of the doped membrane during the initial stage of the desorption. The energy of the subsequent proton exchange step is lowered by lowering the energy required for separating cation and anion of the inorganic oxidant salt. Polyphosphoric acids or polyphosphoric acid cations supply the protons to form a volatile conjugate acid of the inorganic oxidant (i.e., a modified form of the substance of interest). The formation of the active, highly acidic, polyphosphoric acid can be controlled. Because the polycondensation primarily takes place at elevated temperatures, the weakly acidic phosphoric acid is present during the sample collection prior to desorption. This is in contrast to Nafion polymer based ion exchange membranes and conventional sample swabs.

Continuing with the present example, the phosphoric acid loading of a PBI membrane shows a clear stepwise increase in membrane conductivity reflecting the protonation of the first benzimidazole nitrogen followed by protonation of the second nitrogen. This is a basic group titration process that takes time at ambient/room temperature (e.g., hours) as the phosphoric acid has to penetrate the polymer network. The corresponding phosphate anions remain bonded to the doubly protonated imidazole group of the membrane and do not take part in either phosphate or proton diffusion. After the neutralization of the basic groups, further phosphate uptake is observed as a result of hydrogen bonding with the phosphate groups bound to the PBI backbone. In some embodiments, PBI membranes can be loaded with up to 20 phosphate groups per benzimidazole group. Loading above 20 phosphate groups per benzimidazole group causes the membrane to become unstable. The amount of water present in the membrane determines the transfer mechanism of protons and phosphoric acid species and thereby the diffusion rate of these species.

Tables 1a and 1b show proton and phosphate diffusion rates, respectively, for phosphoric acid at relatively low concentrations. The diffusion rate of phosphoric acid inside the PBI copolymer goes up by three orders of magnitude at 240° C. in comparison to the diffusion rate at ambient temperature (25° C.). For instance, the diffusion rate of phosphoric acid at 25° C. is 10⁻⁸ cm² sec⁻¹, while at 240° C. the diffusion rate of phosphoric acid is 10⁻⁵ cm² sec⁻¹ (see Table 1b). Accordingly, the effect of temperature on the diffusion rate of phosphoric acid within a PBI membrane is significant.

TABLE 1a
Proton diffusion rates (cm² sec⁻¹) in benzimidazole
(BI)/phosphoric acid (PA) mixtures.

Temperature (degree Celsius)

| PA/BI Ratio | 240 | 25 |
|---|---|---|
| 9/1 | 8e−6 | 1e−7 |
| 6/1 | 5e−6 | 9e−8 |
| 3/1 | 1e−6 | 1e−8 |

TABLE 1b
Phosphate diffusion rates (cm² sec⁻¹) in benzimidazole
(BI)/phosphoric acid (PA) mixtures.

Temperature (degree Celsius)

| PA/BI Ratio | 240 | 25 |
|---|---|---|
| 9/1 | 1e−5 | 2e−8 |
| 6/1 | 9e−6 | 2e−8 |
| 3/1 | 6e−6 | 3e−9 |

In accordance with the present disclosure, the doped membrane described herein has a generally neutral surface pH of from about 6.0 to about 7.0. This feature is beneficial for safety reasons, presenting minimal risk of chemical (e.g., acid) exposure to anyone handling the doped membrane or to other items contacted by the doped membrane (e.g., sampled luggage, cargo, freight, packages, mail, etc.). In some embodiments, the surface pH of the doped membrane is 6.5. The doped membrane has a thickness of from about 10 μm to about 1000 μm. In some embodiments, the thickness of the doped membrane ranges from about 25 μm to about 900 μm, from about 50 μm to about 750 μm, from about 75 μm to about 500 μm, or from about 100 μm to about 250 μm.

The doped membrane is used to contact and modify a substance of interest to increase its volatility and thereby improve detection selection and/or sensitivity by a trace detection system. Analysis of the modified substance of interest by the trace detection system results in detection and identification of the corresponding un-modified substance of interest that was originally sampled. In some embodiments, the trace detection system includes one or more libraries for identifying substances of interest based on the analysis of the modified, more volatile substance of interest.

In some embodiments, the substance of interest includes at least one of an explosive, an energetic material, a taggant, a narcotic, a toxin, a chemical warfare agent, a biological warfare agent, a pollutant, a pesticide, a toxic industrial chemical, a toxic industrial material, a homemade explosive, a pharmaceutical trace contaminant and combinations thereof. In some embodiments, the substance of interest includes an inorganic salt. For example, the inorganic salt includes at least one of a nitrate, a chlorate, a perchlorate, nitrites, a chlorite, a permanganate, a chromate, a dichromate, a bromate, an iodate, and combinations thereof.

In some embodiments, the doped membrane is incorporated into a sample swab and comprises at least a portion of an outer surface of the sample swab. The sample swab with incorporated doped membrane is used to collect a sample containing at least one substance of interest, and is introduced into a trace detection system inlet. In some embodiments, the substance of interest is chemically modified upon contact with the doped membrane to a more volatile form. The trace detection system includes a desorber which desorbs the sample swab with incorporated doped membrane and releases the modified, more volatile substance of interest for subsequent analysis and detection by the trace detection system.

In some embodiments, the doped membrane is not incorporated into the sample swab. Rather, the doped membrane is positioned within the desorber of the trace detection system. A sample swab is used to collect a sample containing at least one substance of interest and is introduced into the inlet and desorber of the trace detection system. The doped membrane comes into contact with the substance of interest within the desorber by contacting the sample swab (or other sample media). For instance, in some embodiments, the doped membrane is coupled to a mechanical arm that is configured to move the doped membrane for contact with the sample swab. In other embodiments, the mechanical arm is coupled to the sample swab, or is coupled to both the sample swab and doped membrane, such that the doped membrane and the substance of interest on the sample swab come into contact with each other. In some embodiments, a desorption cycle is initiated following contact between the doped membrane and the substance of interest (via contact with the sample swab). In some embodiments, a desorption cycle is initiated after the sample swab has been introduced into the desorber, yet prior to contacting the substance of interest with the doped membrane within the desorber. In these embodiments, the desorption cycle is begun prior to contacting the substance of interest with the doped membrane in order to minimize any potential alteration of the desorption process of other substances of interest (e.g., more conventional explosives) that might be present on the sample swab. Accordingly, the sample swab is contacted with the doped membrane after a suitable amount of time in the desorption cycle and/or after a suitable desorption temperature has been reached.

Once a doped membrane has contacted a substance of interest, the doped membrane and substance of interest are heated in a desorber and the modified, more volatile substance of interest is released for subsequent analysis and detection by the trace detection system. In some embodiments, the substance of interest is released by heating the desorber to a temperature of from about 150° C. to about 270° C.

The substance of interest can be detected using at least one of an ion mobility spectrometer (IMS), an ion trap mobility spectrometer (ITMS), a drift spectrometer (DS), an aspiration ion mobility spectrometer, a non-linear drift spectrometer, a field ion spectrometer (FIS), a radio frequency ion mobility increment spectrometer (IMIS), a field asymmetric ion mobility spectrometer (FAIMS), an ultra-high-field FAIMS, a differential ion mobility spectrometer (DIMS), a differential mobility spectrometer (DMS), a trapped ion mobility spectrometer (TIMS), a traveling wave ion mobility spectrometer, a semiconductor gas sensor, a raman spectrometer, a laser diode detector, a mass spectrometer (MS), a gas chromatograph (GC), an electron capture detector, a photoionization detector, a chemiluminescence-based detector, an electrochemical sensor, an infrared spectrometer, a lab-on-a-chip detector and combinations thereof.

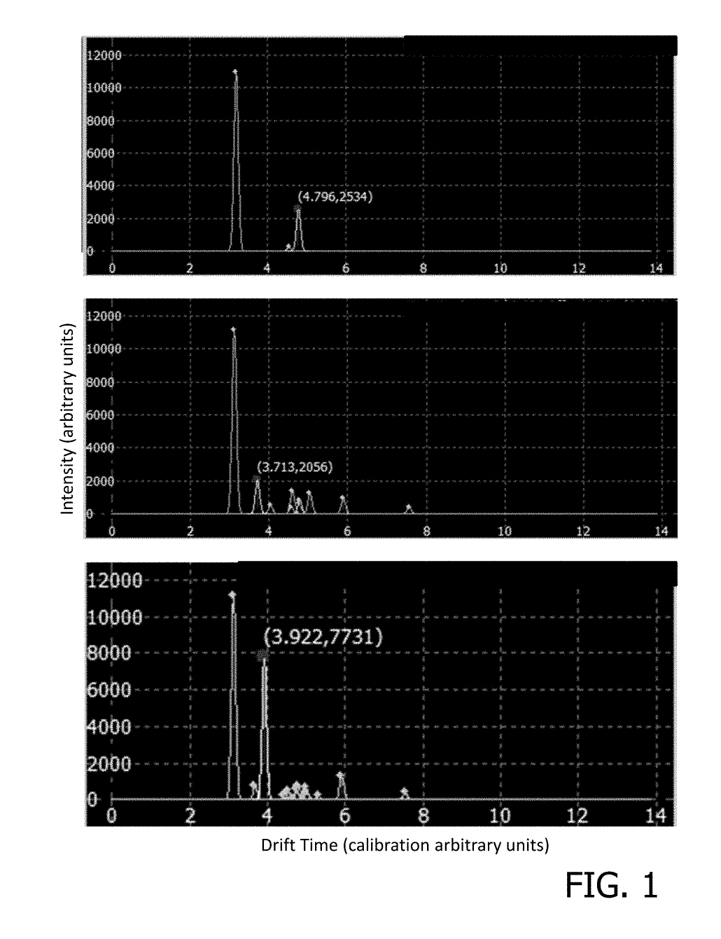

FIG. 1 is an exemplary embodiment of reactant ion peak intensities associated with KClO₃ detection in accordance with the present disclosure. The top spectrum of FIG. 1 shows reactant ion peaks obtained from desorption of a sample swab only, having no substance of interest collected thereon. The middle spectrum of FIG. 1 shows reactant ion peaks obtained from desorption of a sample swab incorporated with a doped membrane as described herein, having no substance of interest collected thereon. The bottom portion of FIG. 1 shows reactant ion peaks obtained from desorption of a sample swab with incorporated doped membrane, and having an inorganic salt, KClO₃ (potassium chlorate), as the substance of interest collected thereon. KClO₃ is known to be a substance of interest found in many home-made explosives (HMEs) which has very low volatility, which makes it difficult to detect using conventional trace detection systems and methods that lack a doped membrane device. In this embodiment, based on calibration for the trace detection system, KClO₃ is known to have a drift time of about 3.94 arbitrary units, or generally within the range of 3.87 and 3.97 units. The top and middle spectra of FIG. 1 exhibit no interfering peaks within the 3.87-3.97 range of interest for KClO₃. Consequently, neither the sample swab alone nor the doped membrane alone contributes any interference to the detection of KClO₃ at 3.87 and 3.97 units. The bottom spectrum shows an intense reactant ion peak for KClO₃ within the range of interest between 3.87 and 3.97 units. Accordingly, KClO₃ is successfully detected using a doped membrane to increase its volatility and improve its detection.

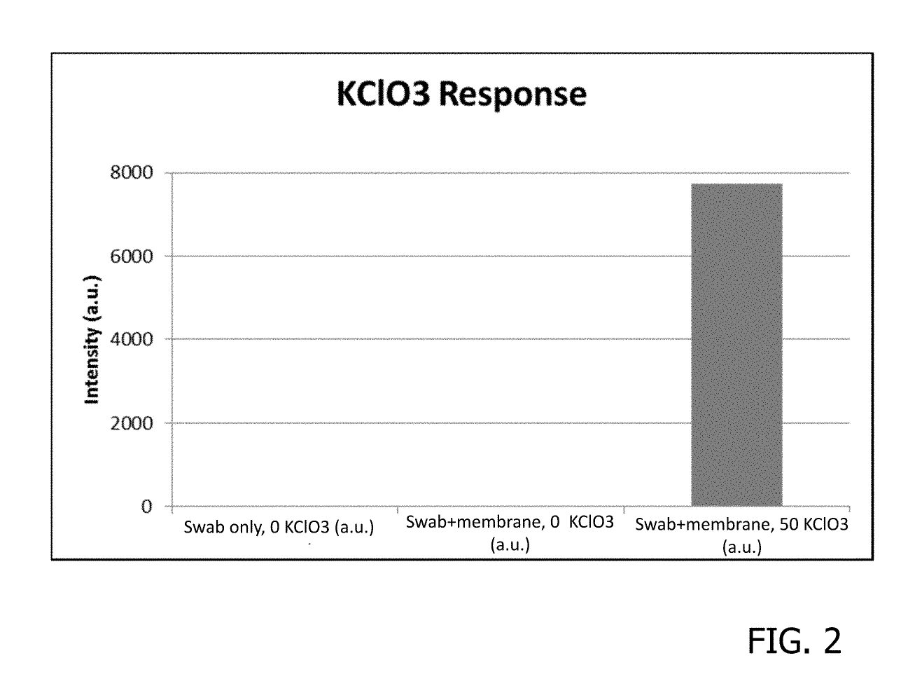

FIG. 2 is an exemplary embodiment of a graphical depiction of the KClO₃ detection response shown in FIG. 1, in accordance with the present disclosure. FIG. 2 summarizes the reactant ion peak response for KClO₃ with respect to a sample swab only, a sample swab with doped membrane, and a sample swab with doped membrane and 50 a.u. KClO₃ as the substance of interest. The left-most and middle columns show no peak intensity (and therefore no interference) within the drift time range of interest between 3.87 and 3.97 arbitrary units. The right-most column shows a strong reactant ion response in the range of interest, attributed to 50 a.u. KClO₃.

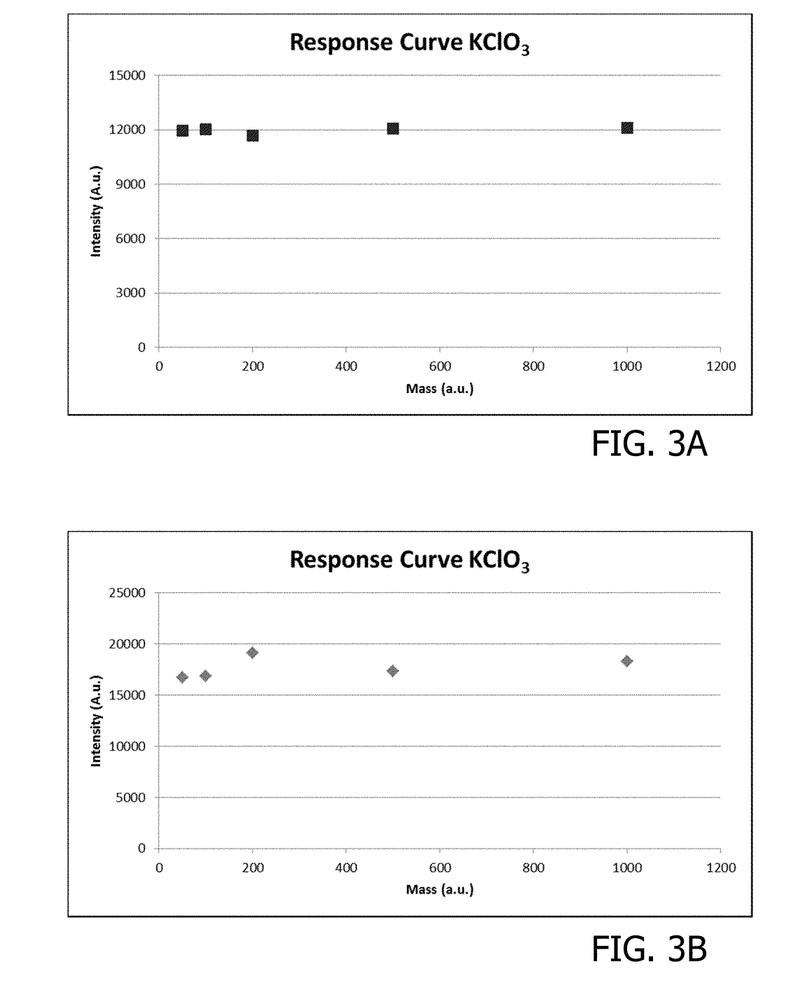

FIGS. 3A and 3B are exemplary embodiments of KClO₃ detection response curves associated with two exemplary doped membranes in accordance with the present disclosure. In these exemplary embodiments, two different doped membranes were contacted with varying masses of KClO₃, ranging from 50 a.u. to 1000 a.u. Both FIGS. 3A and 3B show a saturated intensity response (at approximately 12000 arbitrary units in FIG. 3A, and at approximately 17500 arbitrary units in FIG. 3B) beginning with the lowest mass (50 a.u.). Accordingly, the limit of detection for KClO₃ is much lower than 50 a.u. for each of the two doped membranes utilized.

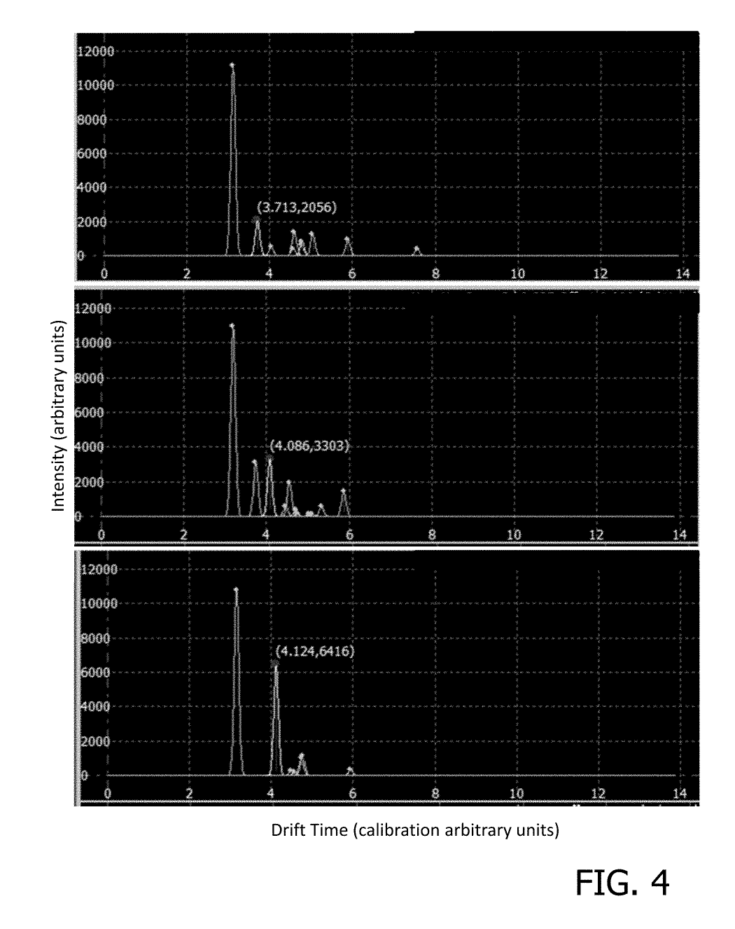

FIG. 4 is an exemplary embodiment of reactant ion peak intensities associated with KClO₄ detection in accordance with the present disclosure. The top spectrum of FIG. 4 shows reactant ion peaks obtained from desorption of a sample swab only, having no substance of interest collected thereon. The middle spectrum of FIG. 4 shows reactant ion peaks obtained from desorption of a sample swab incorporated with a doped membrane as described herein, having an inorganic salt, 200 a.u. of KClO₄ (potassium perchlorate), as the substance of interest collected thereon. The bottom portion of FIG. 4 shows reactant ion peaks obtained from desorption of a sample swab with incorporated doped membrane, and having 5000 a.u. of KClO₄ collected thereon. Similar to KClO₃, KClO₄ is also known to be a substance of interest found in many HMEs and having a very low volatility. In this embodiment, based on calibration for the trace detection system, KClO₄ is known to have a drift time of about 4.09 arbitrary units, or generally within the range of 4.05 and 4.15 units. The top spectrum of FIG. 4 exhibits no interfering peaks within the 4.05-4.15 range of interest for KClO₄. Consequently, the sample swab alone does not contribute any interference to the detection of KClO₄ between 4.05 and 4.15 units. The middle spectrum of FIG. 4 shows a modest intensity response for KClO₄ (approximately over 3000 arbitrary intensity units) between 4.05 and 4.15 drift time units.

The bottom spectrum shows a stronger intensity response for KClO₄ (approximately over 6000 arbitrary intensity units) within the range of interest. Accordingly, KClO₄ is successfully detected using a doped membrane for increasing volatility and improving detection.

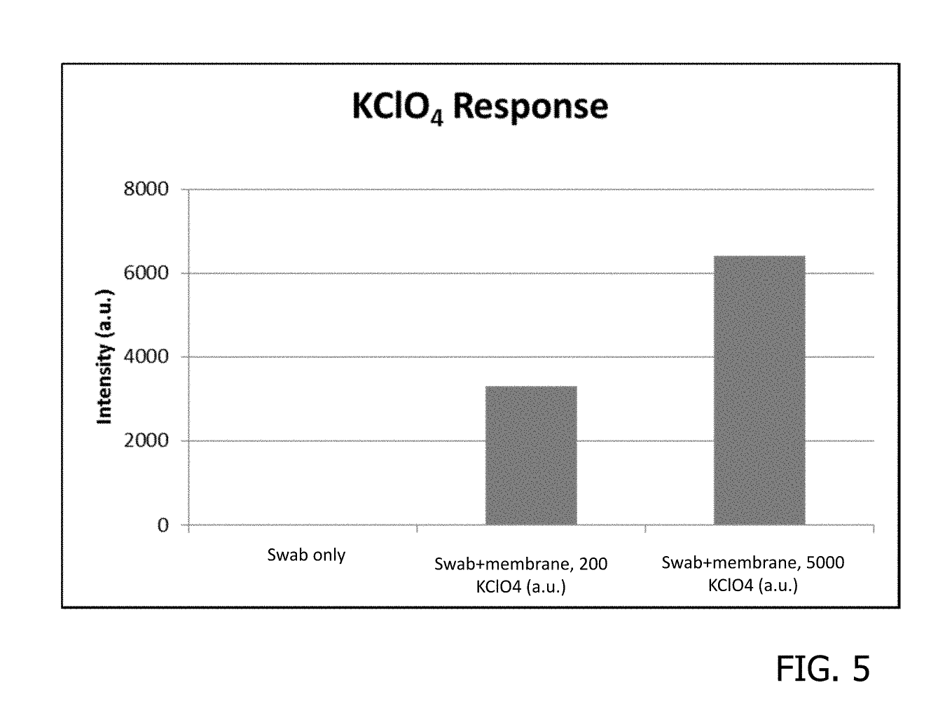

FIG. 5 is an exemplary embodiment of a graphical depiction of KClO₄ detection response shown in FIG. 4, in accordance with the present disclosure. FIG. 5 summarizes the reactant ion peak response for KClO₄ with respect to a sample swab only, a sample swab with doped membrane and 200 a.u. KClO₄ as the substance of interest, and a sample swab with doped membrane and 5000 a.u. KClO₄ as the substance of interest. The left-most column shows no peak intensity (and therefore no interference) within the drift time range of interest between 4.05 and 4.15 arbitrary units. The middle and right-most columns show proportionally responsive intensities for the KClO₄ reactant ion peak in the range of interest, attributed to 200 a.u. KClO₄ and 5000 a.u. KClO₄, respectively.

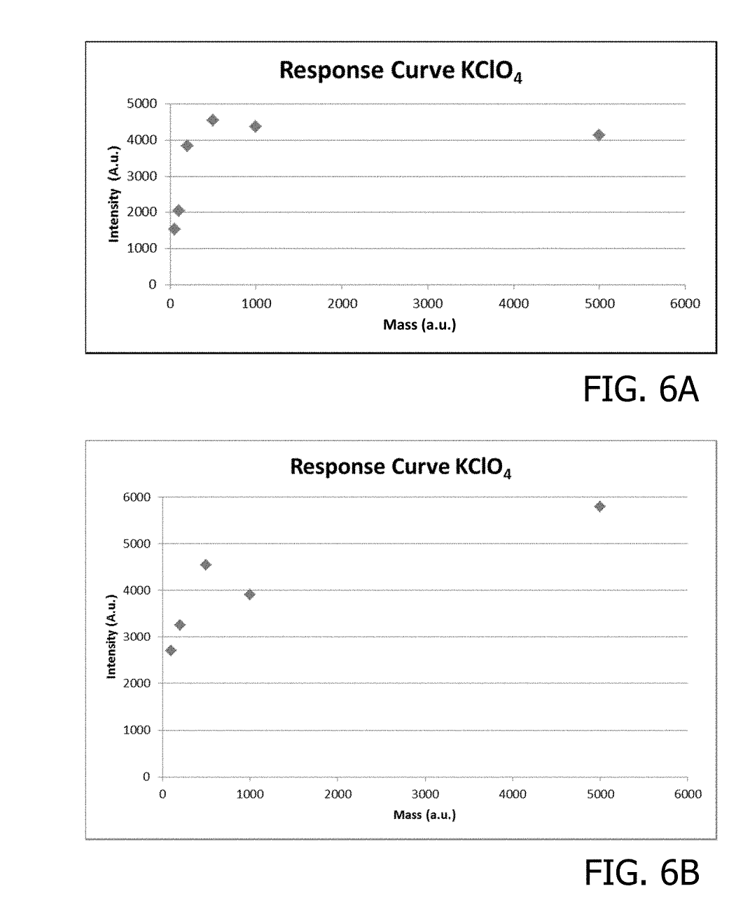

FIGS. 6A and 6B are exemplary embodiments of KClO₄ detection response curves associated with two exemplary doped membranes in accordance with the present disclosure. In these exemplary embodiments, two different doped membranes were contacted with varying masses of KClO₄, ranging from 50 a.u. to 5000 a.u. Both FIGS. 3A and 3B show a dose-dependent up to about 1000 a.u. KClO₄, until reaching their respective saturation point intensity responses (at approximately 4500 arbitrary intensity units in FIG. 6A, and at approximately 5500 arbitrary intensity units in FIG. 6B).

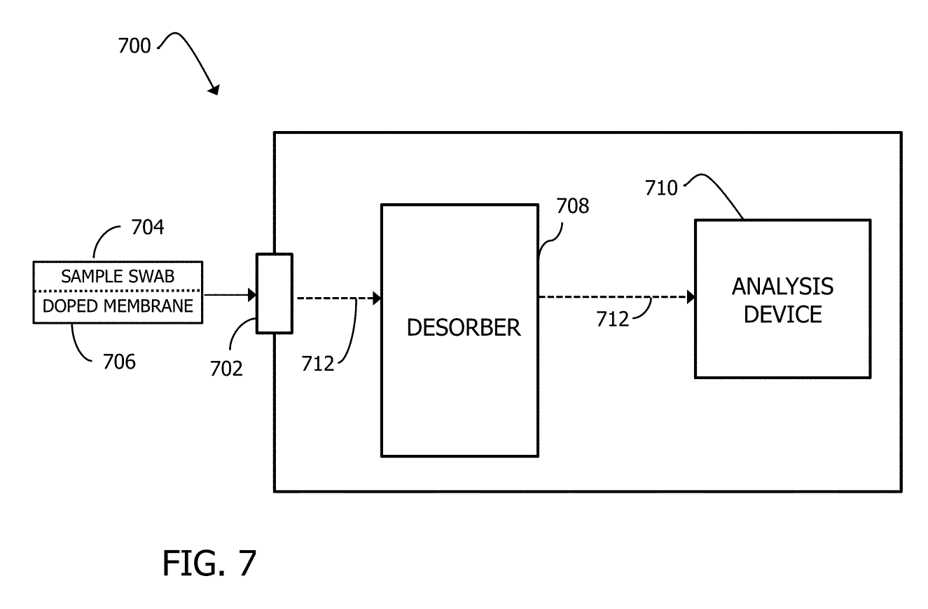

FIG. 7 is an exemplary embodiment of a block diagram of a trace detection system in accordance with the present disclosure. System 700 includes an inlet 702 configured to receive a substance of interest, such as a substance of interest that has been collected on a sample swab 704. Sample swab 704 also includes a doped membrane 706 that comprises at least a portion of an outer surface of sample swab 704. System 700 further includes a desorber 708 and an analysis device 710 in flow communication with one another and also in flow communication with inlet 702, as indicated by arrows 712. Once a sample containing a substance of interest has been collected the sample swab 704 with doped membrane 706, sample swab 704 with doped membrane 706 are inserted into the inlet 702 and subsequently into desorber 708. Desorber 708 is configured to heat the sample swab 704 with doped membrane 706 to release the modified, more volatile substance of interest as described above. In some embodiments, the desorber 708 is heated to a temperature range of from about 150° C. to about 270° C. In some embodiments, the desorber 708 is heated to a temperature range of from about 200° C. to about 250° C. Analysis device 710 is configured to perform an analysis on the substance of interest and detect the substance of interest.

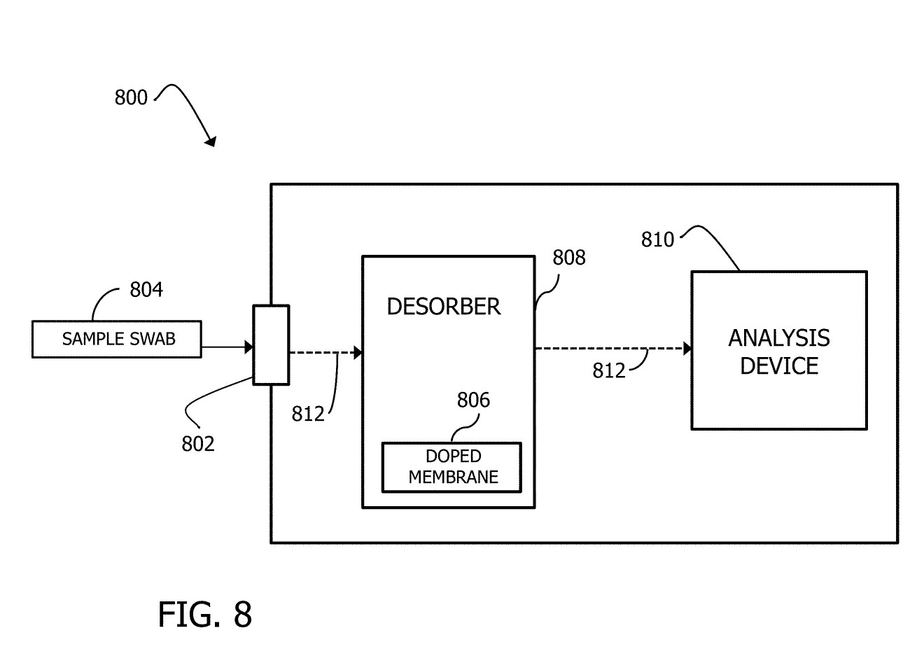

FIG. 8 is another exemplary embodiment of a block diagram of a trace detection system in accordance with the present disclosure. System 800 includes an inlet 802 configured to receive a substance of interest, such as a substance of interest that has been collected on a sample swab 804. Once a sample containing a substance of interest has been collected on sample swab 804, sample swab 804 is inserted into inlet 802. System 800 further includes a desorber 808 and an analysis device 810 in flow communication with one another and also in flow communication with inlet 802, as indicated by arrows 812. A doped membrane 806 is located within the desorber 808. In some embodiments, the doped membrane 806 is coupled to a mechanical arm (not shown) configured to move the doped membrane 806 into contact with sample swab 804 that has been inserted through inlet 802 and subsequently into the desorber 808. In some embodiments, sample swab 804 additionally or alternatively coupled to a mechanical arm configured to move the sample swab 804 into contact with doped membrane 806. Desorber 808 is configured to heat the doped membrane 806 that has contacted the substance of interest via contact with sample swab 804 to release the modified, more volatile substance of interest as described above.

In some embodiments, desorber 808 begins heating the doped membrane 806 following contact between the doped membrane 806 and the substance of interest (via contact with the sample swab 804). In other embodiments, desorber 808 begins heating after the sample swab 804 has been introduced into the desorber 808, yet prior to contacting the substance of interest (collected on sample swab 804) with the doped membrane 806 within the desorber 808. In these embodiments, desorption heating is begun prior to contacting the substance of interest with the doped membrane 806 in order to minimize any potential alteration of the desorption process of other substances of interest (e.g., more conventional explosives) that might be additionally present on the sample swab 804. In some embodiments, the sample swab 804 is contacted with the doped membrane 806 after a suitable amount of time in the desorption cycle and/or after a suitable desorption temperature has been reached. In some embodiments, the desorber 808 is heated to a temperature range of from about 150° C. to about 270° C. In some embodiments, the desorber 808 is heated to a temperature range of from about 200° C. to about 250° C. Analysis device 810 is configured to perform an analysis on the substance of interest and detect the substance of interest.

In some embodiments of the present disclosure, the substance of interest detected by the detection system 700 or 800 includes at least one of an explosive, an energetic material, a taggant, a narcotic, a toxin, a chemical warfare agent, a biological warfare agent, a pollutant, a pesticide, a toxic industrial chemical, a toxic industrial material, a homemade explosive, a pharmaceutical trace contaminant, an inorganic salt, a nitrate, a chlorate, a perchlorate, a nitrite, a chlorite, a permanganate, a chromate, a dichromate, bromates, an iodate, and combinations thereof.

In some embodiments of the present disclosure, the analysis devices 710 and 810 include at least one of an ion mobility spectrometer (IMS), a reverse ion mobility spectrometer, an ion trap mobility spectrometer (ITMS), a drift spectrometer (DS), an aspiration ion mobility spectrometer, a non-linear drift spectrometer, a field ion spectrometer (FIS), a radio frequency ion mobility increment spectrometer (IMIS), a field asymmetric ion mobility spectrometer (FAIMS), an ultra-high-field FAIMS, a differential ion mobility spectrometer (DIMS), a differential mobility spectrometer (DMS), a trapped ion mobility spectrometer (TIMS), a traveling wave ion mobility spectrometer, a semiconductor gas sensor, a raman spectrometer, a laser diode detector, a mass spectrometer (MS), a gas chromatograph (GC), an electron capture detector, a photoionization detector, a chemiluminescence-based detector, an electrochemical sensor, an infrared spectrometer, a lab-on-a-chip detector, and combinations thereof.

Exemplary embodiments of detection systems for determining the presence of substances of interest, and methods of operating such systems are not limited to the specific embodiments described herein, but rather, components of systems and/or steps of the methods may be utilized independently and separately from other components and/or steps described herein. For example, the methods may also be used in combination with other systems requiring determining the presence of substances of interest, and are not limited to practice with only the substance detection systems and methods as described herein. Rather, the exemplary embodiment can be implemented and utilized in connection with many other substance detection applications that are currently configured to determine the presence of substances of interest.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

Some embodiments involve the use of one or more electronic or computing devices. Such devices typically include a processor or controller, such as a general purpose central processing unit (CPU), a graphics processing unit (GPU), a microcontroller, a reduced instruction set computer (RISC) processor, an application specific integrated circuit (ASIC), a programmable logic circuit (PLC), and/or any other circuit or processor capable of executing the functions described herein. The methods described herein may be encoded as executable instructions embodied in a computer readable medium, including, without limitation, a storage device and/or a memory device. Such instructions, when executed by a processor, cause the processor to perform at least a portion of the methods described herein. The above examples are exemplary only, and thus are not intended to limit in any way the definition and/or meaning of the term processor.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A device for detecting a substance of interest, the device comprising a doped membrane, wherein the membrane comprises at least one semi-permeable medium and is doped with at least one acid.

2. The device of claim 1, wherein the at least one acid comprises an organic acid.

3. The device of claim 2, wherein the organic acid comprises at least one of trifluoroacetic acid and formic acid.

4. The device of claim 1, wherein the at least one acid comprises an inorganic acid.

5. The device of claim 4, wherein the inorganic acid comprises at least one of phosphoric acid and polyphosphoric acid.

6. The device of claim 1, wherein the doped membrane has a thickness of from about 10 μm to about 1000 μm, from about 25 μm to about 900 μm, from about 50 μm to about 750 μm, from about 75 μm to about 500 μm, or from about 100 μm to about 250 μm.

7. The device of claim 1, wherein the doped membrane comprises at least a portion of an outer surface of a sample swab.

8. The device of claim 1, wherein the doped membrane comprises a doped ion exchange membrane.

9. The device of claim 1, wherein the at least one semi-permeable medium comprises a polybenzimidazole (PBI) copolymer.

10. The device of claim 1, wherein the semi-permeable medium is doped with the acid at a concentration of from about 0.50 weight percent to about 20 weight percent of the acid.

11. The device of claim 1, wherein the doped membrane has a surface pH of from about 6.0 to about 7.0.

12. The device of claim 1, wherein the substance of interest includes at least one of an explosive, an energetic material, a taggant, a narcotic, a toxin, a chemical warfare agent, a biological warfare agent, a pollutant, a pesticide, a toxic industrial chemical, a toxic industrial material, a homemade explosive, a pharmaceutical trace contaminant and combinations thereof.

13. The device of claim 1, wherein the substance of interest includes an inorganic salt.

14. The device of claim 1, wherein the inorganic salt includes at least one of a nitrate, a chlorate, a perchlorate, nitrites, a chlorite, a permanganate, a chromate, a dichromate, a bromate, an iodate, and combinations thereof.

15. A method for detecting a substance of interest, the method comprising:
contacting a substance of interest with a doped membrane, wherein the membrane comprises at least one semi-permeable medium and is doped with at least one acid;
heating the substance of interest and the doped membrane in a desorber;
performing an analysis of the substance of interest; and
detecting the substance of interest.

16. The method of claim 15, wherein contacting the substance of interest with the doped membrane comprises:
contacting the substance of interest with a sample swab outside of the desorber, wherein the doped membrane comprises at least a portion of an outer surface of the sample swab; and
inserting the sample swab into the desorber.

17. The method of claim 15, wherein contacting the substance of interest with the doped membrane comprises:
contacting the substance of interest on a sample swab;
inserting the sample swab into the desorber; and
contacting the sample swab with the doped membrane inside of the desorber.

18. The method of claim 15, wherein the substance of interest includes at least one of an explosive, an energetic material, a taggant, a narcotic, a toxin, a chemical warfare agent, a biological warfare agent, a pollutant, a pesticide, a toxic industrial chemical, a toxic industrial material, a homemade explosive, a pharmaceutical trace contaminant, an inorganic salt, a nitrate, a chlorate, a perchlorate, a nitrite, a chlorite, a permanganate, a chromate, a dichromate, bromates, an iodate, and combinations thereof.

19. The method of claim 15, wherein heating the substance of interest and the doped membrane in a desorber comprises heating the desorber to a temperature range of from about 150° C. to about 270° C.

20. The method of claim 15, wherein detecting the substance of interest comprises detecting the substance of interest using at least one of an ion mobility spectrometer (IMS), an ion trap mobility spectrometer (ITMS), a drift spectrometer (DS), an aspiration ion mobility spectrometer, a non-linear drift spectrometer, a field ion spectrometer (FIS), a radio frequency ion mobility increment spectrometer (IMIS), a field asymmetric ion mobility spectrometer (FAIMS), an ultra-high-field FAIMS, a differential ion mobility spectrometer (DIMS), a differential mobility spectrometer (DMS), a trapped ion mobility spectrometer (TIMS), a traveling wave ion mobility spectrometer, a semiconductor gas sensor, a raman spectrometer, a laser diode detector, a mass spectrometer (MS), a gas chromatograph (GC), an electron capture detector, a photoionization detector, a chemiluminescence-based detector, an electrochemical sensor, an infrared spectrometer, a lab-on-a-chip detector and combinations thereof.

21. A system for detecting a substance of interest, the system comprising:
an inlet configured to receive a substance of interest;
a doped membrane, wherein the membrane comprises at least one semi-permeable medium and is doped with at least one acid;
a desorber; and
an analysis device coupled in flow communication with the inlet and the desorber, wherein the analysis device is configured to perform an analysis on the substance of interest.

22. The system of claim 21, wherein the doped membrane is located within the desorber.

23. The system of claim 22, further comprising a mechanical arm coupled to the doped membrane and configured to contact the doped membrane with the substance of interest received at the inlet.

24. The system of claim 21, wherein the desorber is configured to heat the doped membrane to a temperature of from about 150° C. to about 270° C.

25. The system of claim 21, wherein the substance of interest includes at least one of an explosive, an energetic material, a taggant, a narcotic, a toxin, a chemical warfare agent, a biological warfare agent, a pollutant, a pesticide, a toxic industrial chemical, a toxic industrial material, a homemade explosive, a pharmaceutical trace contaminant, an inorganic salt, a nitrate, a chlorate, a perchlorate, a nitrite, a chlorite, a permanganate, a chromate, a dichromate, bromates, an iodate, and combinations thereof.

26. The system of claim 21, wherein the analysis device includes at least one of an ion mobility spectrometer (IMS), an ion trap mobility spectrometer (ITMS), a drift spectrometer (DS), an aspiration ion mobility spectrometer, a non-linear drift spectrometer, a field ion spectrometer (FIS), a radio frequency ion mobility increment spectrometer (IMIS), a field asymmetric ion mobility spectrometer (FAIMS), an ultra-high-field FAIMS, a differential ion mobility spectrometer (DIMS), a differential mobility spectrometer (DMS), a trapped ion mobility spectrometer (TIMS), a traveling wave ion mobility spectrometer, a semiconductor gas sensor, a raman spectrometer, a laser diode detector, a mass spectrometer (MS), a gas chromatograph (GC), an electron capture detector, a photoionization detector, a chemiluminescence-based detector, an electrochemical sensor, an infrared spectrometer, a lab-on-a-chip detector and combinations thereof.
